(19) United States
(12) Patent Application Publication
(10) Pub. No.: US 2024/0145068 A1
(43) Pub. Date: May 2, 2024

(54) MEDICAL IMAGE ANALYSIS PLATFORM AND ASSOCIATED METHODS

(71) Applicant: The Trustees of the University of Pennsylvania, Philadelphia, PA (US)

(72) Inventors: Walter R.T. Witschey, Haddonfield, NJ (US); Neil Chatterjee, Chicago, IL (US); Matthew L. MaClean, Chapel Hill, NC (US); Jeffrey T. Duda, Philadelphia, PA (US); James C. Gee, Glandwyn, PA (US); Hersh Sagreiya, Wayne, PA (US); Chales E. Kahn, JR., Milwaukee, WA (US); Ari Borthakur, Philadelphia, PA (US); Ameena Elahi, Philadelphia, PA (US); Kristen Martin, Ewing, NJ (US)

(21) Appl. No.: 18/499,922

(22) Filed: Nov. 1, 2023

Related U.S. Application Data
(60) Provisional application No. 63/421,406, filed on Nov. 1, 2022.

(51) Int. Cl. G16H 30/20; G16H 15/00; G16H 50/70

(52) U.S. Cl. CPC G16H 30/20; G16H 15/00; G16H 50/70

(57) ABSTRACT

Data distribution and analysis systems, and associated methods are described herein. In one aspect, a method for distributing data can include: receiving, by a computing service and from a medical data server, medical image data stored by the PACS; inputting the medical image data into a machine learning model hosted by the computing service, wherein the computing service allows users to upload machine learning models and configure corresponding machine learning models to interface with the medical data server; generating, by the machine learning model, additional biometric data corresponding to the medical image data; and sending the additional biometric data to the medical data server, wherein the additional biometric data and the medical image data is incorporated into an electronic health record (EHR).

### CROSS-REFERENCE TO RELATED APPLICATIONS

The present application claims priority to and the benefit of U.S. patent application No. 63/421,406, “Medical Image Analysis Platform and Associated Methods” (filed Nov. 1, 2022), the entirety of which application is incorporated herein by reference for any and all purposes.

### TECHNICAL FIELD

The disclosed technology relates to the field of data distribution and analysis, and in particular, data distribution and analysis systems for medical imaging data.

### BACKGROUND

Artificial Intelligence (AI) has the potential to reduce labor, lower costs, and improve diagnostic accuracy for medical professionals, including radiologists. This can benefit both patients and radiologists and is becoming a valuable tool to provide safer treatments and better outcomes for patients as well as streamline operations. AI, more specifically deep-learning (DL) algorithms, a subset of machine learning (ML) computing, is rapidly finding use in diagnostic image analyses.

In order to optimize a radiologist's time, an AI solution ideally can be available on a picture archiving and communication system (PACS) before the radiologist reads the study, which can mitigate potential for report addendums. But most existing PACS systems cannot process computationally-intense AI algorithms. Some AI vendors suggest that processing be performed on-site versus in the cloud, but this requires an expensive (e.g., over $15 k), dedicated image processing computer system. Advent of cloud-based computing services (e.g. Amazon Web Services, Microsoft Azure, Google Cloud, etc.) allows practitioners and researchers with minimal infrastructure and tools to develop and deploy ML algorithms at scale. There exists a need for a platform capable of managing medical imaging data and supporting AI-based analysis configured to process the medical imaging data.

### SUMMARY

Data distribution and analysis systems, and associated methods are described herein. In one aspect, a method for distributing data can include: receiving, by a computing service and from a medical data server, medical image data stored by the PACS; inputting the medical image data into a machine learning model hosted by the computing service, wherein the computing service allows users to upload machine learning models and configure corresponding machine learning models to interface with the medical data server; generating, by the machine learning model, additional biometric data corresponding to the medical image data; and sending the additional biometric data to the medical data server, wherein the additional biometric data and the medical image data is incorporated into an electronic health record (EHR).

### BRIEF DESCRIPTION OF THE DRAWINGS

For the purpose of illustrating the invention, there is shown in the drawings a form that is presently preferred; it being understood, however, that this invention is not limited to the precise arrangements and instrumentalities shown.

FIG. 1 depicts a data distribution system according to the present disclosure;

FIG. 2 depicts a process flow for analyzing medical image data according to the present disclosure;

FIG. 3 depicts a process flow for analyzing medical image data according to the present disclosure;

FIG. 4 depicts a process flow for analyzing medical image data according to the present disclosure;

FIG. 5 depicts a process flow for analyzing medical image data according to the present disclosure; and

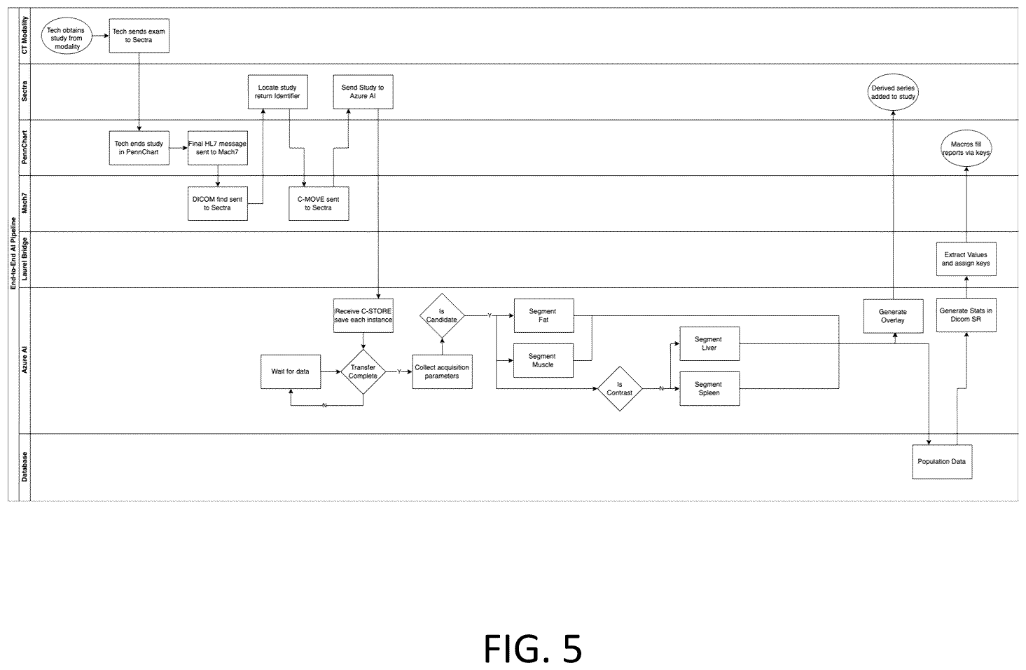

FIG. 6 depicts a system capable of performing the processes described herein.

FIG. 7 depicts an example of the data distribution system receiving CT imaging data, analyzing the CT imaging data via a machine learning model hosted or managed by the system, and generating data that is included in a patient report (e.g., a patient radiology report).

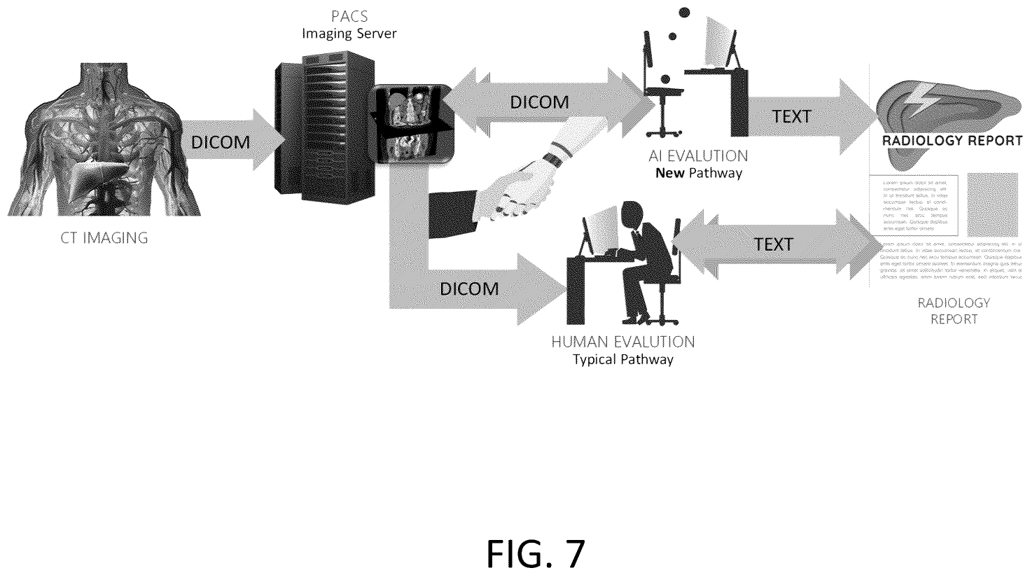

FIG. 8 depicts data flow into a data distribution system according to the present disclosure the system can be capable of receiving different types of data across different types of environments. The various data can be sent to the system, which may be capable of analyzing different data type (e.g., either separately or in combination), and can provide detailed, comprehensive analyses for a patient (e.g., based on the machine learning model provided).

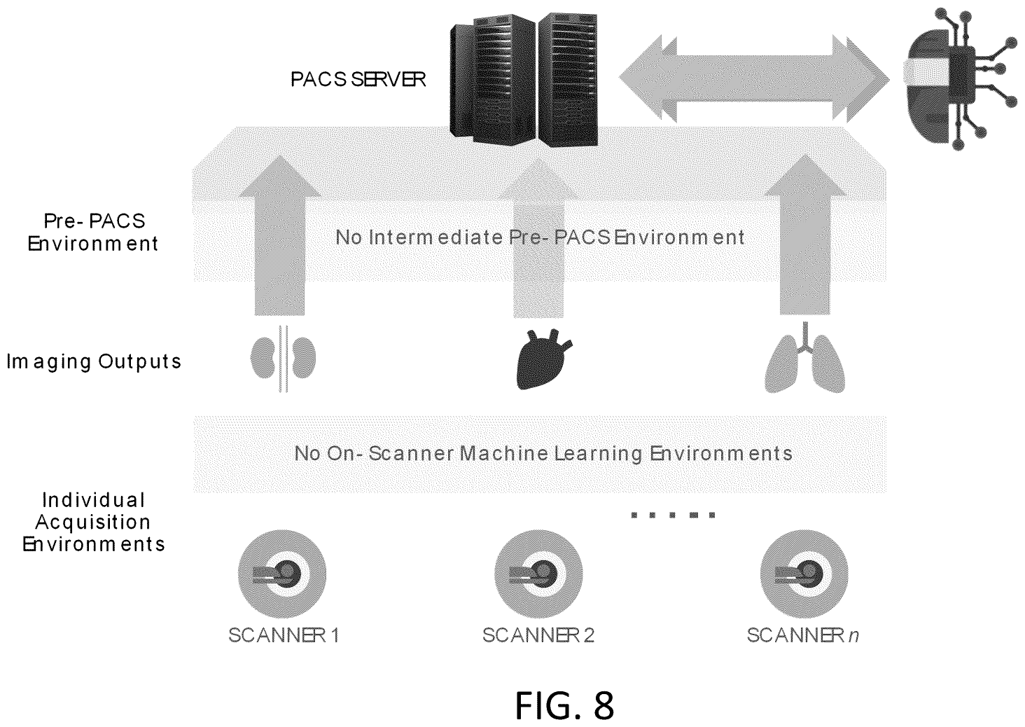

FIG. 9 depicts example abdominal CT scans analyzed by machine learning models of a data distribution system according to the present disclosure.

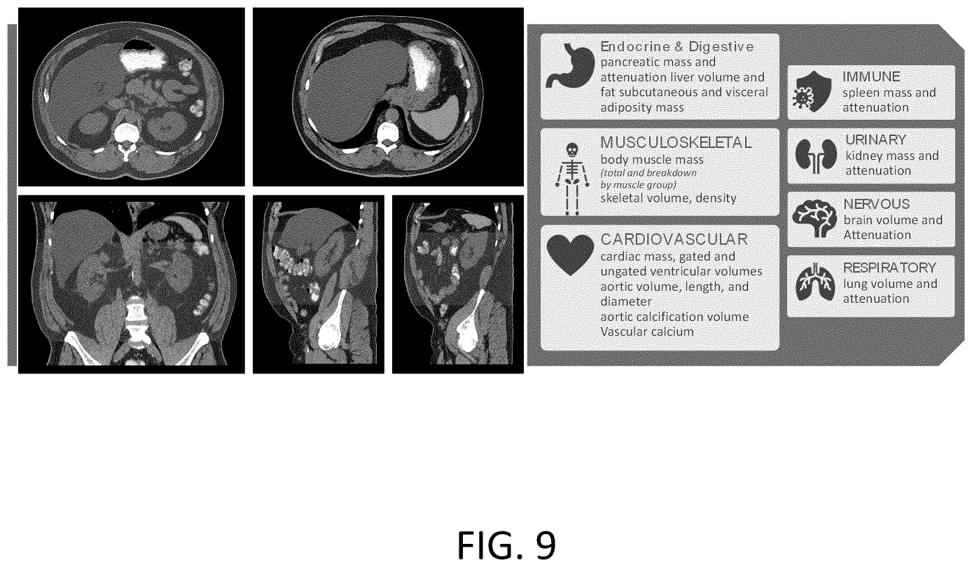

FIG. 10 depicts a graph of a number of patients analyzed over time by a data distribution system according to the present disclosure.

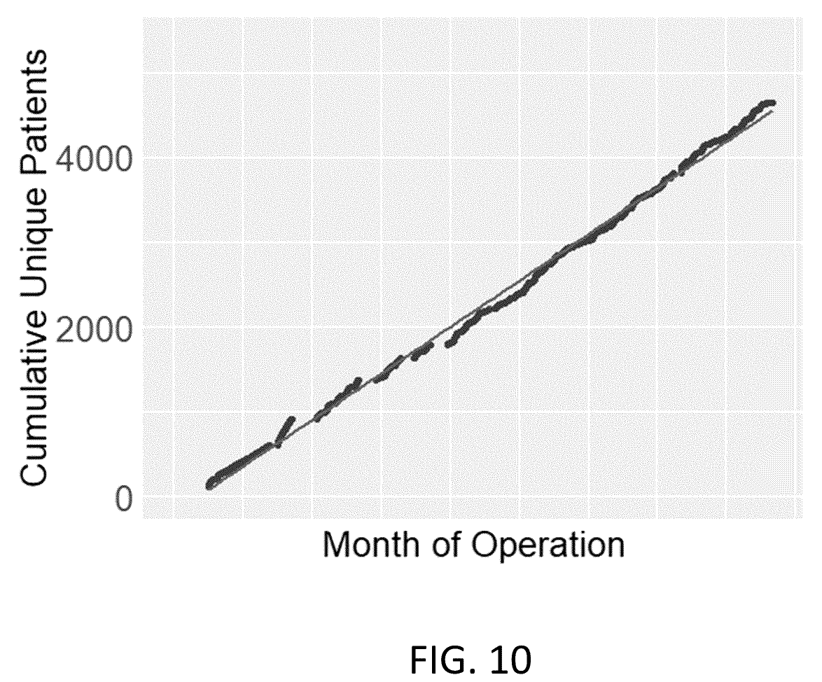

FIG. 11 depicts a graph of analysis time for patient data over time by a data distribution system according to the present disclosure.

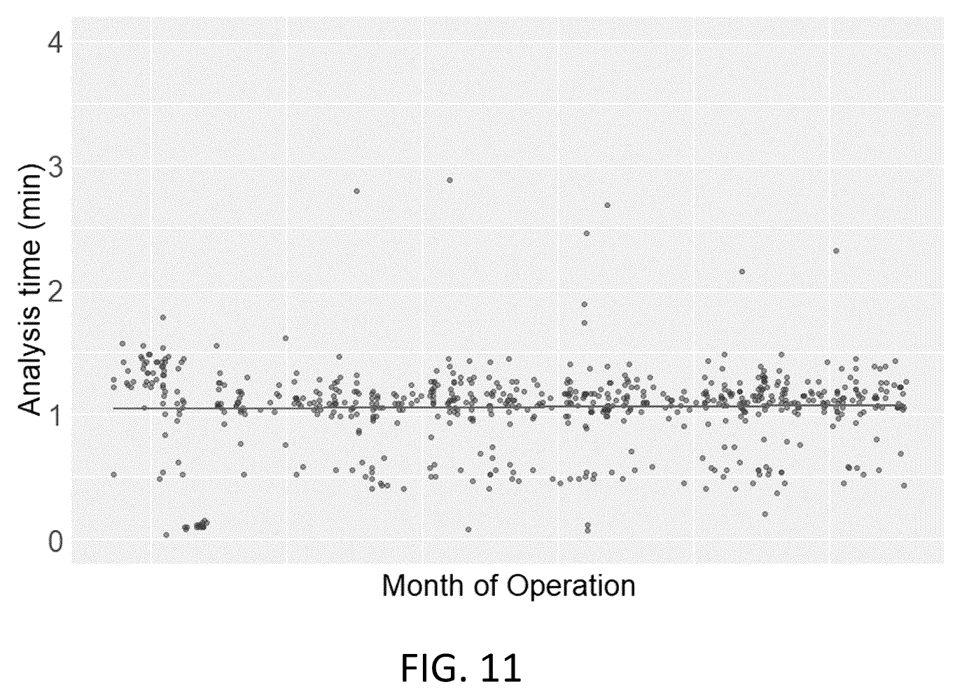

FIG. 12 depicts graphs of automated screening for fatty liver disease using a data distribution system according to the present disclosure. The left graph is a graph of liver attenuation over time determined by the data distribution system; and the right graph is a histogram of frequency of determined patients having elevated liver fat over the respective value of liver attenuation.

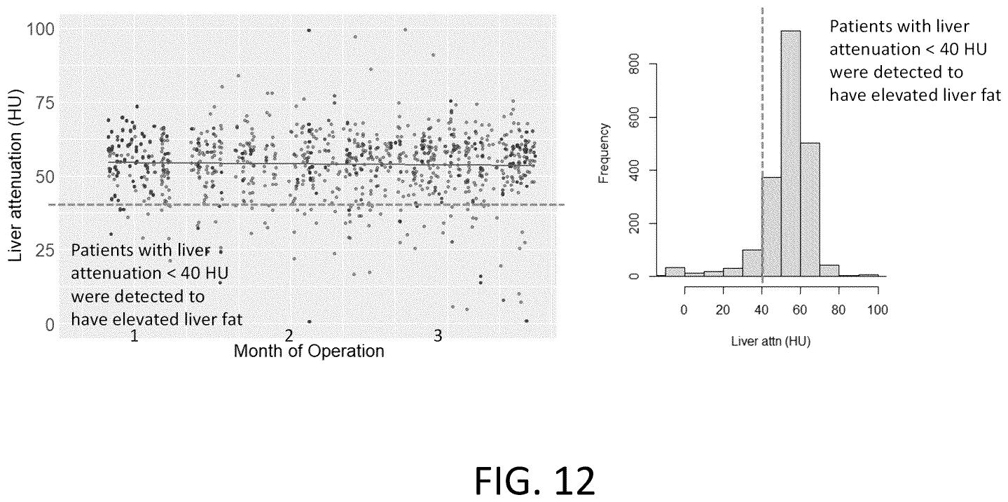

FIG. 13 depicts graphs of determining HbAlc and diabetic status of patients based on synthetic blood panels analyzed by a data distribution system according to the present disclosure.

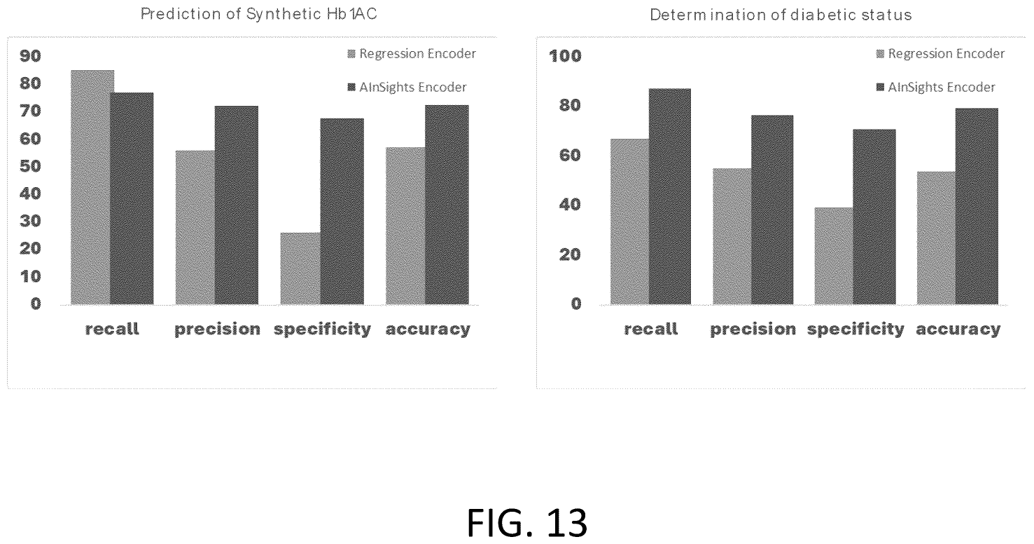

FIG. 14 depicts a graph of determining pathologic age-related muscle loss (sarcopenia) in patients by a data distribution system according to the present disclosure.

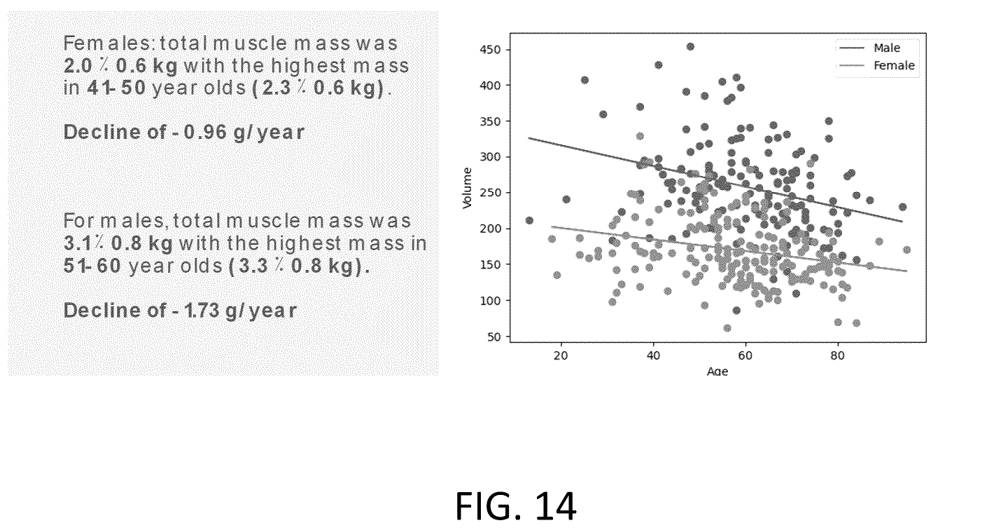

### DETAILED DESCRIPTION OF ILLUSTRATIVE EMBODIMENTS

The present disclosure may be understood more readily by reference to the following detailed description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable, and it should be understood that steps may be performed in any order. Any documents cited herein are incorporated by reference in their entireties for any and all purposes.

It is to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any subcombination. Further, reference to values stated in ranges include each and every value within that range. In addition, the term “comprising” should be understood as having its standard, open-ended meaning, but also as encompassing “consisting” as well. For example, a device that comprises Part A and Part B may include parts in addition to Part A and Part B, but may also be formed only from Part A and Part B.

Data distribution and analysis systems, and associated methods are described herein. A data distribution platform can receive medical image data, such as radiological images (X-rays, CT scans, MRIs, PET scans, ultrasounds, and the like) can be stored and transferred from a picture archiving and communication system (PACS). The image data can be sent to a cloud computing network capable of hosting or communicating with artificial intelligence (AI) models configured to analyze the image data and provide additional biometric data from the analyzing. The additional biometric data can be fed back to the PACS, where the biometric data can be incorporated into an electronic health record for a given patient. Medical professionals, such as radiologists, can thus receive additional data corresponding to the health of a patient, without having to perform additional labor. Additionally, the system can be configured to incorporate a number of different AI models capable of providing different analyses. The models can thus be tailored to particular patients, health conditions, demographics, and the like.

The data distribution system can include a near real-time de-identified medical image pipeline for machine learning (ML) researchers to obtain and process clinical images and patient data using services from a PACS using any cloud services provider (e.g., Amazon Web Services (AWS)). In addition, the world of ML code developers readily share code on websites such as GitHub and Kaggle, providing an opportunity for rapid testing and implementation of various AI models capable of being incorporated into the data distribution system.

Additionally, the system can access these and other ML algorithms to generate inferences on newly-acquired patient data in near real-time using a platform developed with custom-written software. Based on different clinical sub-specialties, different ML algorithms and automated tasks can be performed to provide actionable data/images back for radiologists' review, and also to remove manual processes, e.g. detecting critical events and tracking regulatory metrics for reporting.

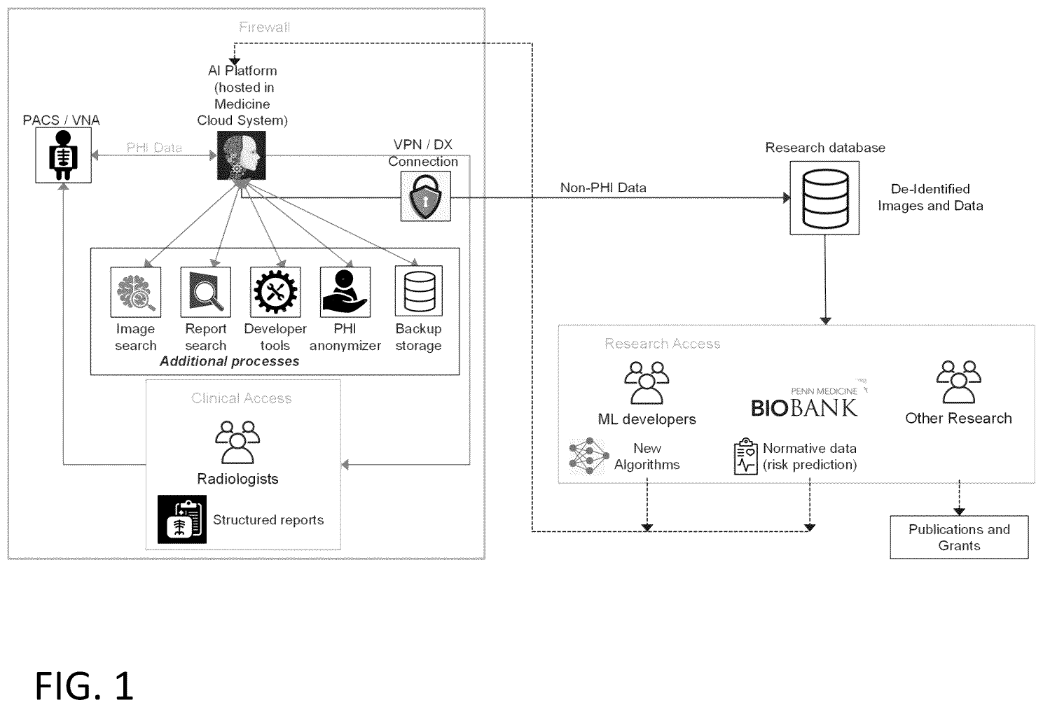

A feature of the data distribution system is the development of a cloud-based medical image analysis platform (cMIAP) (an example of which is depicted in FIG. 1), which begins by ingesting images obtained from PACS that triggers a set of steps and actions to be performed. The cMIAP can be a distributed computing service, which can be managed across multiple computers or nodes. The computers can be connected to one another via a wide area network (WAN) if located in different geographic locations. In some cases, the distributed computing service can be implemented by one or more virtual machines (VMs) which can reside in the distributed computers.

A process for retrieving and analyzing medical image data via the system described herein can include: 1) The cMIAP can run in parallel to current radiology workflow without requiring any changes except to connect to the existing PACS systems; 2) the cMIAP can process each image and its associated patient data inside a computing system, such as Penn Med Academic Computing System (PMACS); an image can be sent to a server of the system (e.g., a personal health information—(PHI)-compliant server, such as a Penn Medicine PHI-compliant server) for automatic ingestion into the processing pipeline; an image de-identifying algorithms can de-identify medical text using, for example, Natural Language Processing (e.g. Comprehend Medical service in AWS) to detect and extract text from image files; image filters can be applied to the image if necessary; the cMIAP can detect and tokenize PHI found within the image itself, which can be stored in the cMIAP (e.g., inside a PMACS firewall), along with other metadata; the token mappings can be stored in a secured database (e.g., a DynamoDB service in AWS) so that the images can be re-identified at the PHI-compliant server; and a search service (e.g. ElasticSearch in AWS) can detect and record medically relevant entities within the images and metadata for a searchable database of clinical images.

The cMIAP can transfer non-PHI data to storage (e.g., Amazon S3 storage) where de-identified images are accessible to ML researchers and clinical data analyses pipelines. The transferring can occur periodically, such as via a push communication. In some cases, the transferring can occur in an aperiodic process, such as if storage sends a pull notification or request for image data to be sent from the cMIAP to the PACS or PMACS. In some cases, PACS can scan for new medical image data, which, if found, can be transferred for analysis. In some cases, medical image data can be requested from PACs or PMACS based on a triggering event, such as user input received for executing a machine learning model for analyzing medical image data. If images are retrieved for ML algorithms development, then then the images can be accessed by a research account. If the images are part of a prediction for augmenting clinical workflow, then the images can be accessible by a clinical account.

Validated ML algorithms can be transferred, as needed, from research to clinical accounts. Finally, the prediction image and data are transferred back to the PHI-compliant server (e.g., PMACS-based) server where they are re-identified and inserted back into PACS. The pipeline described above can be shared with other image analysis developers for future improvements or additional analysis data.

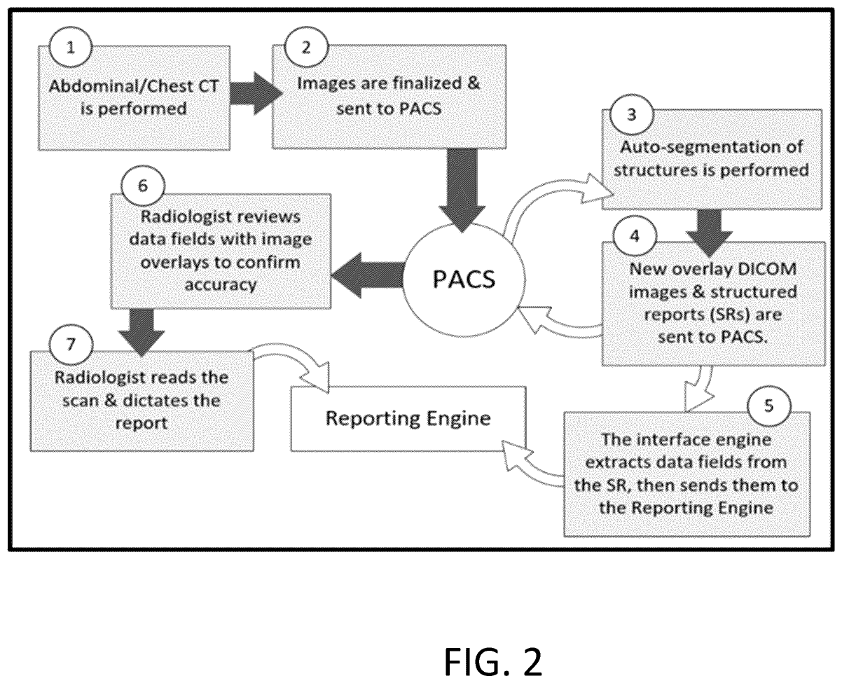

The system described herein can also implement an AI model to generate additional biometric data based on the medical image data received from the PACS. FIG. 2 depicts a pipeline for generating additional biometric data according to the present disclosure.

Once imaging is completed and finalized, the patient's images can be sent to PACS where they can be sent to a server for processing (e.g., segmentation, overlay and structured reporting of image-based phenotypes). This “inference engine” can then send these results back into PACS for a radiologist to add to their report, and can extract preset data fields to combine with the radiologist's report in the “reporting engine” part of the system. For example, electronic health reports can be stored as Digital Imaging and Communications in Medicine (DICOM) format. The reporting engine and/or inference engine can generate metadata based on results from the machine learning model output. The metadata can be formatted as a DICOM field, such as a DICOM tag, to be added to a DICOM folder associated with a particular medical image. The DICOM tag(s) can be sent to the PACS, where the metadata can be added to the DICOM file.

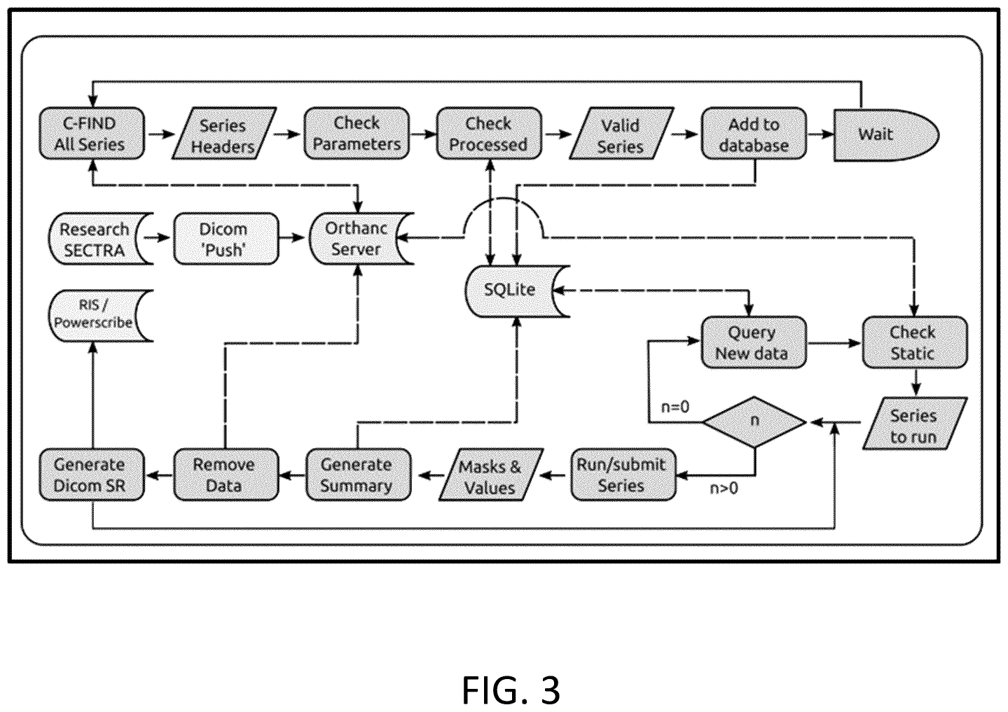

The inference engine can reside in the cloud or can reside in a local imaging server. FIG. 3 (right) depicts a software pipeline that resides in a PMACS-based server to perform several tasks. Briefly, a local imaging server (“Orthanc Server”) can receive medical images from a clinical PACS server (“Research Sectra”). Two applications can run concurrently shown in the top pipeline and the bottom pipeline. The top application can compute images stored on the Orthanc Server and stores metadata in a database (SQLite). The bottom application can analyze medical images by retrieving pixel data from Orthanc. The Orthanc server can communicate back and forth with a cloud service (e.g., AWS DataSync) depicted in FIG. 1 during the model development phase.

The system can also include a user interface (UI) for managing the cMIAP. The UI can allow for a user to manage various aspects of the cMIAP. For example, the UI can allow for a user to configure a given machine learning model hosted by the cMIAP. In some case, the UI can provide access for a user to train a hosted machine learning model (e.g., supervised training). In some cases, the UI can provide user access to selecting a machine learning model for implementing medical imaging analysis. In some cases, the UI can provide user access to select particular images for analysis via a hosted machine learning model (e.g., indicated via DICOM tags). In some cases, the UI can provide user access to configure or select datatypes to be added in an EHR (e.g., selecting different DICOM tags or information types). In some cases, the UI can provide user access to select a particular medical data server (e.g., PACS) to retrieve medical image data from. In some cases, the UI can provide user access to select a particular server or network to retrieve population or normative data from.

The system described herein can be a flexible, unified solution to allow radiology organizations to quickly deploy AI-based clinical algorithms and optimize their workflow processes in support of increased productivity and diagnostic confidence. This can be achieved without significant upfront hardware costs.

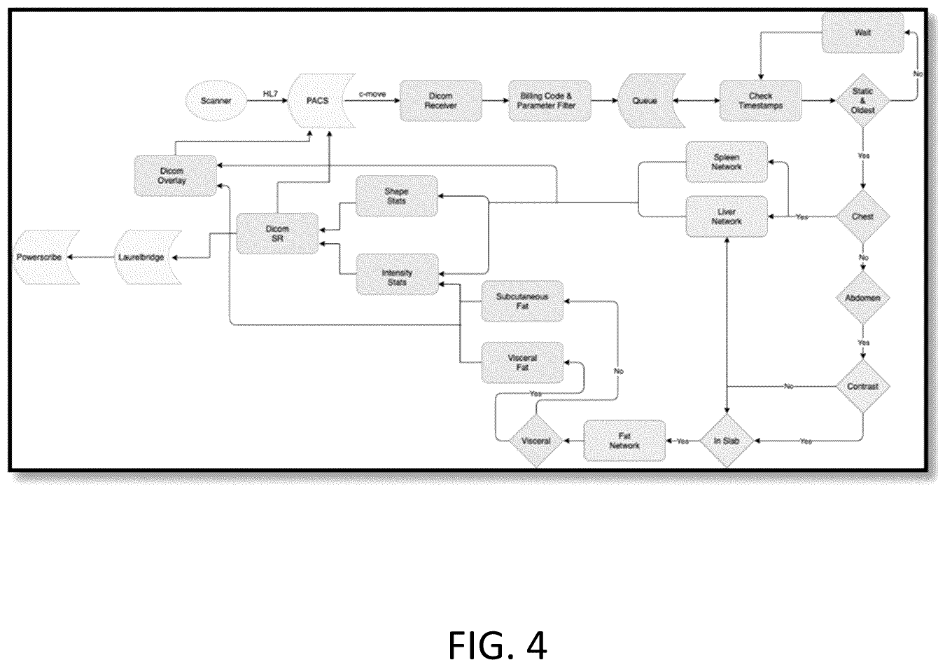

In some cases, the AI model incorporated into the data distribution system can provide abdominal inferencing data. The model can include a classification algorithm that curates unenhanced CT images, can segment the spleen to provide a liver attenuation reference, and can quantify fat from chest CTs with partial liver views in addition to abdominal/pelvic scans. A flow diagram of the process is depicted in FIG. 4. Initially, images can move from scanner to PACS as part of routine radiology workflow, which are then intercepted by a decision diamond triggering the workflow described herein. Patients' CT images of chest or abdomen both with or without contrast can be sent to separate AI networks for extraction of imaging phenotypes into structured reports (SR) and DICOM color overlays that are sent to Powerscribe and PACS, respectively, for radiologists to review and finalize into their patients' radiology reports.

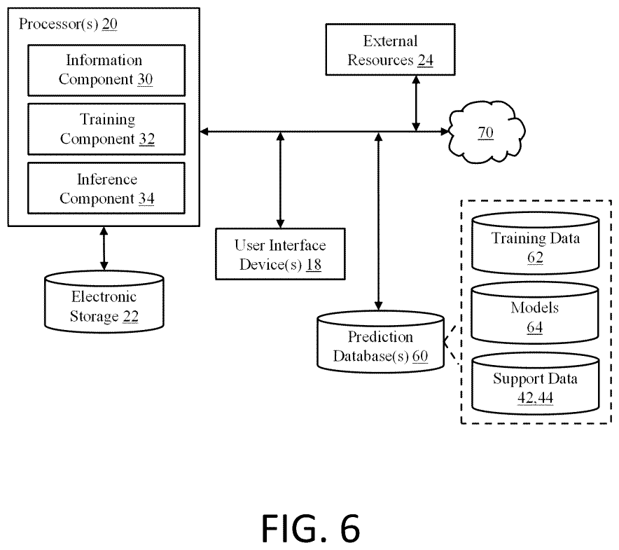

FIG. 6 depicts a system 600 capable of storing medical and/or analyzing medical image data according to the present disclosure. The system 600 can be an example of a cloud computing network, such as the cloud computing network described with reference to FIG. 1.

The system 600 can include one or more AI models 64. Some embodiments of models 64 can comprise a CNN. A CNN can comprise an input and an output layer, as well as multiple hidden layers. The hidden layers of a CNN typically comprise a series of convolutional layers that convolve with a multiplication or other dot product. The activation function is commonly a RELU layer, and is subsequently followed by additional convolutions such as pooling layers, fully connected layers and normalization layers, referred to as hidden layers because their inputs and outputs are masked by the activation function and final convolution.

The CNN computes an output value by applying a specific function to the input values coming from the receptive field in the previous layer. The function that is applied to the input values is determined by a vector of weights and a bias (typically real numbers). Learning, in a neural network, progresses by making iterative adjustments to these biases and weights. The vector of weights and the bias are called filters and represent particular features of the input (e.g., a particular shape).

Training component 32 of FIG. 6 can prepare one or more prediction models 64 to generate predictions. Models 64 can analyze made predictions against a reference set of data called the validation set. In some use cases, the reference outputs can be provided as input to the prediction models, which the prediction model can utilize to determine whether its predictions are accurate, to determine the level of accuracy or completeness with respect to the validation set data, or to make other determinations. Such determinations can be utilized by the prediction models to improve the accuracy or completeness of their predictions. In another use case, accuracy or completeness indications with respect to the prediction models' predictions can be provided to the prediction model, which, in turn, can utilize the accuracy or completeness indications to improve the accuracy or completeness of its predictions with respect to input data. For example, a labeled training dataset can enable model improvement. That is, the training model can use a validation set of data to iterate over model parameters until the point where it arrives at a final set of parameters/weights to use in the model.

A model implementing a neural network can be trained using training data obtained by information component 30 from training data 62 storage/database. The training data can include many attributes of objects or other portions of a content item. For example, this training data obtained from prediction database 60 of FIG. 6 can comprise hundreds, thousands, or even many millions of pieces of information (e.g., images or other sensed data) describing objects. The dataset can be split between training, validation, and test sets in any suitable fashion. For example, some embodiments can use about 60% or 80% of the images for training or validation, and the other about 40% or 20% can be used for validation or testing. In another example, training component 32 can randomly split the labelled images, the exact ratio of training versus test data varying throughout. When a satisfactory model is found, training component 32 can, e.g., train it on 95% of the training data and validate it further on the remaining 5%.

The validation set can be a subset of the training data, which is kept hidden from the model to test accuracy of the model. The test set can be a dataset, which is new to the model to test accuracy of the model. The training dataset used to train prediction models 64 can leverage, via inference component 34, an SQL server, and/or a Pivotal Greenplum database for data storage and extraction purposes.

In some embodiments, training component 32 can be configured to obtain training data from any suitable source, via electronic storage 22, external resources 24 (e.g., which can include sensors), network 70, and/or user interface (UI) device(s) 18. The training data can comprise captured images, and/or other discrete instances of sensed information.

In some embodiments, training component 32 can enable one or more prediction models 64 to be trained. The training of the neural networks can be performed via several iterations. For each training iteration, a classification prediction (e.g., output of a layer) of the neural network(s) can be determined and compared to the corresponding, known classification. As such, the neural networks can be configured to receive at least a portion of the training data as an input feature space. Once trained, the model(s) can be stored in database/storage 64 of prediction database 60, as shown in FIG. 6, and then used to classify samples of images based on attributes.

Electronic storage 22 of FIG. 6 comprises electronic storage media that electronically stores information. The electronic storage media of electronic storage 22 can comprise system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 22 can be (in whole or in part) a separate component within system 10, or electronic storage 22 can be provided (in whole or in part) integrally with one or more other components of system 10 (e.g., a user interface device 18, processor 20, etc.). In some embodiments, electronic storage 22 can be located in a server together with processor 20, in a server that is part of external resources 24, in user interface devices 18, and/or in other locations. Electronic storage 22 can comprise a memory controller and one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Electronic storage 22 can store software algorithms, information obtained and/or determined by processor 20, information received via user interface devices 18 and/or other external computing systems, information received from external resources 24, and/or other information that enables system 10 to function as described herein.

External resources 24 can include sources of information (e.g., databases, websites, etc.), external entities participating with system 10, one or more servers outside of system 10, a network, electronic storage, equipment related to Wi-Fi technology, equipment related to Bluetooth® technology, data entry devices, a power supply (e.g., battery powered or line-power connected, such as directly to 110 volts AC or indirectly via AC/DC conversion), a transmit/receive element (e.g., an antenna configured to transmit and/or receive wireless signals), a network interface controller (NIC), a display controller, a set of graphics processing units (GPUs), and/or other resources. In some implementations, some or all of the functionality attributed herein to external resources 24 can be provided by other components or resources included in system 10. Processor 20, external resources 24, UI device 18, electronic storage 22, a network, and/or other components of system 10 can be configured to communicate with each other via wired and/or wireless connections, such as a network (e.g., a local area network (LAN), the Internet, a wide area network (WAN), a radio access network (RAN), a public switched telephone network (PSTN), etc.), cellular technology (e.g., GSM, UMTS, LTE, 5G, etc.), Wi-Fi technology, another wireless communications link (e.g., radio frequency (RF), microwave, IR, ultraviolet (UV), visible light, cm wave, mm wave, etc.), a base station, and/or other resources.

UI device(s) 18 of system 10 can be configured to provide an interface between one or more users and system 10. UI devices 18 are configured to provide information to and/or receive information from the one or more users. UI devices 18 include a UI and/or other components. The UI can be and/or include a graphical UI (GUI) configured to present views and/or fields configured to receive entry and/or selection with respect to particular functionality of system 10, and/or provide and/or receive other information. In some embodiments, the UI of UI devices 18 can include a plurality of separate interfaces associated with processors 20 and/or other components of system 10. Examples of interface devices suitable for inclusion in UI device 18 include a touch screen, a keypad, touch sensitive and/or physical buttons, switches, a keyboard, knobs, levers, a display, speakers, a microphone, an indicator light, an audible alarm, a printer, and/or other interface devices. The present disclosure also contemplates that UI devices 18 include a removable storage interface. In this example, information can be loaded into UI devices 18 from removable storage (e.g., a smart card, a flash drive, a removable disk) that enables users to customize the implementation of UI devices 18.

In some embodiments, UI devices 18 are configured to provide a UI, processing capabilities, databases, and/or electronic storage to system 10. As such, UI devices 18 can include processors 20, electronic storage 22, external resources 24, and/or other components of system 10. In some embodiments, UI devices 18 are connected to a network (e.g., the Internet). In some embodiments, UI devices 18 do not include processor 20, electronic storage 22, external resources 24, and/or other components of system 10, but instead communicate with these components via dedicated lines, a bus, a switch, network, or other communication means. The communication can be wireless or wired. In some embodiments, UI devices 18 are laptops, desktop computers, smartphones, tablet computers, and/or other UI devices.

Data and content can be exchanged between the various components of the system 10 through a communication interface and communication paths using any one of a number of communications protocols. In one example, data can be exchanged employing a protocol used for communicating data across a packet-switched internetwork using, for example, the Internet Protocol Suite, also referred to as TCP/IP. The data and content can be delivered using datagrams (or packets) from the source host to the destination host solely based on their addresses. For this purpose the Internet Protocol (IP) defines addressing methods and structures for datagram encapsulation. Of course other protocols also can be used. Examples of an Internet protocol include Internet Protocol Version 4 (IPv4) and Internet Protocol Version 6 (IPv6).

In some embodiments, processor(s) 20 can form part (e.g., in a same or separate housing) of a user device, a consumer electronics device, a mobile phone, a smartphone, a personal data assistant, a digital tablet/pad computer, a wearable device (e.g., watch), augmented reality (AR) googles, virtual reality (VR) googles, a reflective display, a personal computer, a laptop computer, a notebook computer, a work station, a server, a high performance computer (HPC), a vehicle (e.g., embedded computer, such as in a dashboard or in front of a seated occupant of a car or plane), a game or entertainment system, a set-top-box, a monitor, a television (TV), a panel, a space craft, or any other device. In some embodiments, processor 20 is configured to provide information processing capabilities in system 10. Processor 20 can comprise one or more of a digital processor, an analog processor, a digital circuit designed to process information, an analog circuit designed to process information, a state machine, and/or other mechanisms for electronically processing information. Although processor 20 is shown in FIG. 6 as a single entity, this is for illustrative purposes only. In some embodiments, processor 20 can comprise a plurality of processing units. These processing units can be physically located within the same device (e.g., a server), or processor 20 can represent processing functionality of a plurality of devices operating in coordination (e.g., one or more servers, user interface devices 18, devices that are part of external resources 24, electronic storage 22, and/or other devices).

As shown in FIG. 6, processor 20 is configured via machine-readable instructions to execute one or more computer program components. The computer program components can comprise one or more of information component 30, training component 32, inference component 34, and/or other components. Processor 20 can be configured to execute components 30, 32, and/or 34 by: software; hardware; firmware; some combination of software, hardware, and/or firmware; and/or other mechanisms for configuring processing capabilities on processor 20.

It should be appreciated that although components 30, 32, and 34 are illustrated in FIG. 6 as being co-located within a single processing unit, in embodiments in which processor 20 comprises multiple processing units, one or more of components 30, 32, and/or 34 can be located remotely from the other components. For example, in some embodiments, each of processor components 30, 32, and 34 can comprise a separate and distinct set of processors. The description of the functionality provided by the different components 30, 32, and/or 34 described below is for illustrative purposes, and is not intended to be limiting, as any of components 30, 32, and/or 34 can provide more or less functionality than is described. For example, one or more of components 30, 32, and/or 34 can be eliminated, and some or all of its functionality can be provided by other components 30, 32, and/or 34. As another example, processor 20 can be configured to execute one or more additional components that can perform some or all of the functionality attributed below to one of components 30, 32, and/or 34.

### EXEMPLARY EMBODIMENTS

The following embodiments are exemplary only and do not serve to limit the scope of the present disclosure of the appended claims. It should be understood that any part of any one or more Embodiments can be combined with any part of any other one or more Embodiments.

### Embodiment 1

A method for distributing data, comprising: receiving, by a computing service and from a medical data server, medical image data stored by the PACS; inputting the medical image data into a machine learning model hosted by the computing service, wherein the computing service allows users to upload machine learning models and configure corresponding machine learning models to interface with the medical data server; generating, by the machine learning model, additional biometric data corresponding to the medical image data; and sending the additional biometric data to the medical data server, wherein the additional biometric data and the medical image data is incorporated into an electronic health record (EHR).

### Embodiment 2

The method of Embodiment 1, wherein the medical image data comprises a radiological image.

### Embodiment 3

The method of any of Embodiments 1-2, wherein the medical image data is stored in a Digital Imaging and Communications in Medicine (DICOM) format.

### Embodiment 4

The method of any of Embodiments 1-3, further comprising: receiving population data associated with the medical image data; generating normative medical data from the population data and the additional biometric data; and sending the normative medical data to the medical data server, wherein the normative medical data is incorporated into the EHR.

### Embodiment 5

The method of any of Embodiments 1-4, wherein the medical image data comprises anonymized data, wherein the method further comprises: receiving a token from the medical data server associated with the medical image data; and sending, to the medical data server, the token with the additional biometric data.

### Embodiment 6

The method of any of Embodiments 1-5, wherein the machine learning model comprises a convolutional neural network (CNN).

### Embodiment 7

The method of any of Embodiments 1-6, further comprising: selecting the medical image data for inputting into the machine learning model based on an image type of the medical image data.

### Embodiment 8

The method of any of Embodiments 1-7, further comprising: receiving, by the cloud computing network and from the medical data server, a plurality of medical image datasets; inputting the plurality of medical image datasets into the machine learning model; and training the machine learning model with the plurality of medical image datasets.

### Embodiment 9

An apparatus comprising a processor, memory, and computer-executable instructions stored in the memory that, when executed by the processor, cause the apparatus to perform the method of any of Embodiments 1-8.

### Embodiment 10

A system for data distribution, comprising: a medical data server configured to: receive medical image data; store the medical image data as an electronic health record; and send the medical data; a computing service configured to: receive the medical image data from the medical data server; input the medical image data into an machine learning model hosted by the computing service, wherein the computing service allows users to upload machine learning models and configure corresponding machine learning models to interface with the medical data server; generate, by the machine learning model, additional biometric data corresponding to the medical image data; and send the additional biometric data to the medical data server.

### Embodiment 11

The system of Embodiment 10, wherein the medical data server is further configured to store the additional biometric data in an electronic health record (EHR).

### Embodiment 12

The system of any of Embodiments 10-11, wherein the medical image data comprises a radiological image, and wherein the system further comprises a camera configured to capture the radiological image.

### Embodiment 13

The system of any of Embodiments 10-12, wherein the camera comprises a positron emission tomography (PET) camera, an ultrasound camera, a magnetic resonance imaging (MRI) camera, an X-ray camera, or a computerized tomography (CT) camera.

### Embodiment 14

The system of any of Embodiments 10-13, wherein the cloud computing network is further configured to: receive population data associated with the medical image data; generate normative medical data from the population data and the additional biometric data; and send the normative medical data to the medical data server.

### Embodiment 15

The system of any of Embodiments 10-14, wherein the medical data server is further configured to store the normative medical data in an electronic health record (EHR).

What is claimed:

1. A method for distributing data, comprising:
receiving, by a computing service and from a medical data server, medical image data stored by the PACS;
inputting the medical image data into a machine learning model hosted by the computing service, wherein the computing service allows users to upload machine learning models and configure corresponding machine learning models to interface with the medical data server;
generating, by the machine learning model, additional biometric data corresponding to the medical image data; and
sending the additional biometric data to the medical data server, wherein the additional biometric data and the medical image data is incorporated into an electronic health record (EHR).

2. The method of claim 1, wherein the medical image data comprises a radiological image.

3. The method of claim 1, wherein the medical image data is stored in a Digital Imaging and Communications in Medicine (DICOM) format.

4. The method of claim 1, further comprising:
receiving population data associated with the medical image data;
generating normative medical data from the population data and the additional biometric data; and
sending the normative medical data to the medical data server, wherein the normative medical data is incorporated into the EHR.

5. The method of claim 1, wherein the medical image data comprises anonymized data, wherein the method further comprises:
receiving a token from the medical data server associated with the medical image data; and
sending, to the medical data server, the token with the additional biometric data.

6. The method of claim 1, wherein the machine learning model comprises a convolutional neural network (CNN).

7. The method of claim 1, further comprising:
selecting the medical image data for inputting into the machine learning model based on an image type of the medical image data.

8. The method of claim 1, further comprising:
receiving, by the computing service and from the medical data server, a plurality of medical image datasets;
inputting the plurality of medical image datasets into the machine learning model; and
training the machine learning model with the plurality of medical image datasets.

9. An apparatus comprising a processor, memory, and computer-executable instructions stored in the memory that, when executed by the processor, cause the apparatus to perform the method of claim 1.

10. A system for data distribution, comprising:
a medical data server configured to:
receive medical image data;
store the medical image data as an electronic health record; and
send the medical data;
a computing service configured to:
receive the medical image data from the medical data server;
input the medical image data into an machine learning model hosted by the computing service, wherein the computing service allows users to upload machine learning models and configure corresponding machine learning models to interface with the medical data server;
generate, by the machine learning model, additional biometric data corresponding to the medical image data; and
send the additional biometric data to the medical data server.

11. The system of claim 10, wherein the medical data server is further configured to store the additional biometric data in an electronic Health Record (EHR).

12. The system of claim 10, wherein the medical image data comprises a radiological image, and wherein the system further comprises a camera configured to capture the radiological image.

13. The system of claim 12, wherein the camera comprises a positron emission tomography (PET) camera, an ultrasound camera, a magnetic resonance imaging (MRI) camera, an X-ray camera, or a computerized tomography (CT) camera.

14. The system of claim 10, wherein the computing service is further configured to:
receive population data associated with the medical image data;
generate normative medical data from the population data and the additional biometric data; and
send the normative medical data to the medical data server.

15. The system of claim 14, wherein the medical data server is further configured to store the normative medical data in an electronic health record (EHR).
